(19) United States
(12) Patent Application Publication
(10) Pub. No.: US 2020/0196910 A1
(43) Pub. Date: Jun. 25, 2020

(54) MONITORING APPARATUS OF MOTION PATTERN

(71) Applicant: LIFETECH SCIENTIFIC (SHENZHEN) CO., LTD., Shenzhen (CN)

(72) Inventors: Lepeng ZENG, Shenzhen (CN); Wangcai LIAO, Shenzhen (CN)

(73) Assignee: LIFETECH SCIENTIFIC (SHENZHEN) CO., LTD., Shenzhen (CN)

(21) Appl. No.: 16/721,017

(22) Filed: Dec. 19, 2019

(30) Foreign Application Priority Data
Dec. 24, 2018 (CN) 201811581348.9

(51) Int. Cl. A61B 5/11; A61B 5/00

(52) U.S. Cl. CPC A61B 5/1101; A61B 5/0024; A61B 5/1123; A61B 2562/0247; A61B 5/6807; A61B 5/4082; A61B 2562/0219; A61B 5/681

(57) ABSTRACT

This invention is related to motion pattern monitoring apparatus, comprising: measurement module and monitoring unit; with the said measurement modules being placed in the part of the body of the patient to be monitored; with the monitoring unit being carried by the patient; with the monitoring unit connected by wireless communication to the said measurement modules; with the said measurement modules acquiring the motion signals from the patient and transmitting the motion signals to the monitoring unit, with the monitoring unit analyzing the motion signals to detect the peaks of the signals and to measure the peak amplitudes and peak intervals between two consecutive peaks, and accordingly to identify the motion pattern based on the said peak information. The invention uses the peak amplitudes and peak intervals to detect the motion patterns.

### TECHNOLOGY

The invention disclosure is related to smart monitoring technology, specifically related to the monitoring apparatus of motion pattern in patients with Parkinson's Disease.

### BACKGROUND

Parkinson's Disease (hereafter call PD) is a long-term degenerative disorder of the central nervous apparatus that mainly affects the motor apparatus. As shown in FIG. 1, in the early phase of PD the noticeable symptoms include shaking, rigidity, slowness of movement and difficulty with walking. The monitoring of the motion patterns in PD victims and suspects of PD is of significance in the treatment and control of PD. Traditional method is to count the steps with a wearable device such as a smart wrist band in order to acquire the patient's motion pattern. However, the wearable device constrains the information specifically to the place where the device is bound. Therefore, the effects of the motion pattern monitoring, and thus the usefulness is limited.

### DESCRIPTION OF THE DISCLOSURE

To overcome the limitations of the traditional monitoring devices, a new monitoring apparatus is invented.

This motion pattern monitoring apparatus comprises measurement modules and monitoring unit;

Wherein the said measurement modules are placed in the places where measurement is taken; wherein the said monitoring unit is a handhold unit carried by the patient; wherein the monitoring unit is connected through wireless communication methods to the measurement modules;

Wherein the measurement modules acquire the motion related signals from the patient and transmit the said motion signals to the said monitoring unit;

Wherein the monitoring unit identifies the peaks of the said acquired motion signals, obtains the peak amplitudes and the intervals between two consecutive peaks, and detects the motion patterns based on the said peak amplitudes and peak intervals.

In one of the embodiments, the said monitoring unit uses the said peak amplitudes and peak intervals to identify the status of tremor and/or walking.

As one of the embodiments, the said monitoring unit obtains the number of tremor and number of steps in a preset time window, and uses these numbers to detect the motion status of the patient in the preset time window.

As another one of the embodiments, the said monitoring unit declares the detection of: 1) walking and tremor status in the said time window in the said patient, if the said monitoring unit finds that the number of steps in the said time window is greater than or equal to the preset first step threshold and the number of tremors in the said time window is greater than or equal to the preset first tremor threshold; 2) walking status only in the said time window in the said patient, if the said monitoring unit finds that the number of steps in the said time window is greater than or equal to the preset first step threshold and the number of tremors in the said time window is less than the preset first tremor threshold; 3) tremor status only in the said time window in the said patient, if the said monitoring unit finds that the number of steps in the said time window is less than the preset first step threshold and the number of tremors in the said time window is greater than or equal to the preset first tremor threshold; 4) slowness of movement status in the said time window in the said patient, if the said monitoring unit finds that the number of steps in the said time window is greater than the preset second step threshold and less than the preset first step threshold, and the number of tremors in the said time window is greater than the preset second tremor threshold and less than the preset third tremor threshold; and 5) resting status in the said time window in the said patient, if the said monitoring unit finds that the number of steps in the said time window is less than or equal to the preset second step threshold and the number of tremors in the said time window is less than or equal to the preset second tremor threshold.

In one of the embodiments, the said measurement modules comprise a first measurement module;

wherein the said first measurement module is placed on the wrist of the patient; wherein the said first measurement module is connected to the said monitoring unit via wireless communication methods;

wherein the said first measurement module acquires the 3-D acceleration signals from the wrist of the patient and transmits the said 3-D acceleration signals to the said monitoring unit.

As one of the embodiments, the said first measurement module comprises a first microcontroller, a first wireless communication module and a 3-D accelerometer;

wherein the said 3-D accelerometer is connected to the said first wireless communication module via the said first microcontroller;

wherein the acceleration signals acquired by the said 3-D accelerometer at the said patient's wrist are transmitted to the said first microcontroller;

wherein the said first microcontroller transmits the said acceleration signals to the said monitoring unit via the said first wireless communication module.

As one of the embodiments, the said first measurement module also comprises a first ADC; wherein the said first ADC connects the 3-D accelerometer and the said first microcontroller.

In one of the embodiments, the said measurement modules comprise a second measurement module;

wherein the said second measurement module is placed under the plantar of the said patient; wherein the said second measurement module is connected to the said monitoring unit via wireless communication methods;

wherein the said second measurement module acquires the plantar pressure signals and transmits the plantar pressure signals to the said monitoring unit.

As one of the embodiments, the said second measurement module comprises a second microcontroller, a second wireless communication module and multiple pressure sensors;

wherein each of the pressure sensors is connected to the said second wireless communication module via the said second microcontroller;

wherein the plantar pressure signals acquired by the plantar pressure sensors worn by the said patient are transmitted to said second microcontroller;

wherein the said second microcontroller transmits the said plantar pressure signals to the said monitoring unit.

As one of the embodiments, the said second measurement module also comprises a second ADC; wherein the inputs of the said second ADC are connected to the plantar pressure sensors; wherein the outputs of the said second ADC are connected to the said second microcontroller.

The above-described motion pattern monitoring apparatus allows the measurement modules to be placed at the locations where the motion related signals can be more easily captured such that the motion pattern specific to the PD patient can be obtained. Because of the high signal-to-noise ratio of the acquired motion signals, the peak detection can be conducted with high performance and thus the peak amplitudes and peak intervals can be measured more accurately, which ensures the high performance in the motion pattern identification.

### FIGURE DESCRIPTION

FIG. 1 is diagrammatic sketch of PD in early phase;

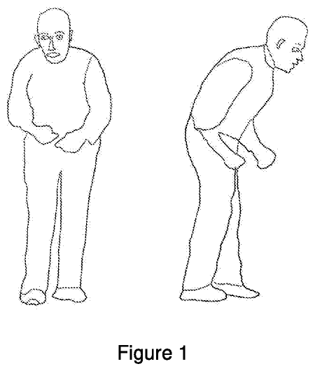

FIG. 2 is the schematic diagram of one embodiment of the structure of the PD motion pattern monitoring apparatus;

FIG. 3 is the flowchart of one embodiment of the motion pattern detection process;

FIG. 4 is the schematic diagram of one embodiment of the structure of the first measurement module;

FIG. 5 is an exemplary diagram of acceleration signals from one embodiment of wrist bend;

FIG. 6 is an exemplary diagram of acceleration signals from one embodiment of wrist bend when the tremor was deliberately suppressed;

FIG. 7 is the schematic diagram of one embodiment of the structure of the first measurement module;

FIG. 8 is the schematic diagram of one embodiment of the structure of the second measurement module;

FIG. 9 is the schematic diagram of one embodiment of the plantar pressure sensors;

FIG. 10 is the schematic diagram of one embodiment of the structure of the second measurement module;

FIG. 11 is the schematic diagram of one embodiment of the mobile device;

FIG. 12 is the schematic diagram of one embodiment of process of the motion pattern monitoring apparatus for PD patients.

### EMBODIMENTS

In order to make the description of the disclosure and the advantage of the implementations of the disclosure easy to understand, the figures are used in the following detailed description. One should understand that the descriptions below are only for the purposes of explanation of the disclosure rather than limiting the protection to cover the disclosure.

It is necessary that the terms used in this disclosure “first/second/third” are only for the convenience of description rather than for specific order. Therefore, under normal condition the orders are interchangeable, which means that other orders different from the one used in the exemplary embodiment can also be implemented.

Terms used in the embodiments, such as “comprising” and “consisting” and any of their variations are to cover the non-exclusive inclusion. For example, a process, a method, a apparatus, a product or a device that consists of a series of steps or modules doesn't mean that it is limited to the already listed steps or module, instead, it can selectively include the steps or modules that are not listed; or selectively includes the other steps or modules that are intrinsic to these processes, methods, products or devices.

“Embodiment” used in the disclosure means that specific characteristic, structure or feature that described in the embodiment can be included in at least one of the embodiments of the application. This term appearing in different places neither means the same embodiment, nor means independent or backup embodiments exclusive to other embodiments. One skilled in the art should understand either explicitly or implicitly it as that the embodiments described in the disclosure ban be combined with other embodiments.

The term “multiple” used in the disclosure refers to two or more than two; “and/or” refers to connection relationship, in which three relationships exist. For instance, A and/or B, may mean one of the three conditions: only A, A and B, and only B. Symbol “/” usually is used to indicate “or” relationship.

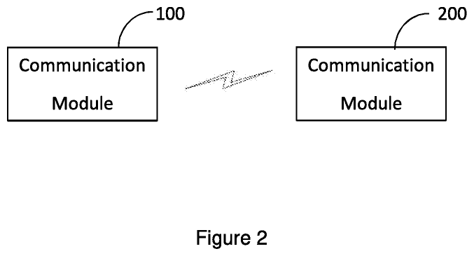

Take FIG. 2 for example. FIG. 2 is an embodiment illustrating the schematic diagram of the structure of the motion pattern monitoring apparatus, comprising: measurement module 100 and monitoring unit 200.

Wherein the said measurement module 100 is placed in the part of the body where patient's motion characteristics are monitored; wherein the said monitoring unit 200 is carried by the patient; wherein the monitoring unit 200 communicates with the measurement module via the wireless communication connection;

Wherein the said measurement module 100 acquires the motion signals from the patient and transmits the signals to the said monitoring unit 200;

Wherein the said monitoring unit 200 detects each peak from the said motion signals and obtains the peak amplitudes and the peak intervals between two consecutive peaks, and further identifies the motion patterns based on the peak amplitudes and peak intervals.

The above-mentioned measurement module 100 may consist of sensors that can acquire motion signals from the patient in real-time and wireless communication module, in order to transmit the acquired motion signals by the said sensors to monitoring unit 200. The above-mentioned monitoring unit 200 may consist of smart device, such as a smart phone, a tablet and/or laptop computer; wherein the said monitoring unit 200 can output the peak amplitudes, peak intervals and the subsequently derived motion patterns from the motion signal to the display such that the patient can see the monitored motion pattern results in real-time. The above-mentioned measurement module 100 may contain various types of sensors in order to acquire the information of motion from the patient; the above-mentioned measurement modules can be placed in relevant parts of the patient's body to acquire the reliable motion signals. For instance, the said measurement module 100 may include the first measurement module to obtain the limb's acceleration signals and/or the second measurement module to acquire plantar pressure signals; wherein the said first measurement module can be placed on the limbs to acquire the limb acceleration signals; while the said second measurement module can be placed under plantar to acquire the plantar pressure signals.

The said patient can be a PD victim (i.e. one with Parkinson's Disease) or suspect of PD who needs his motion patterns to be monitored. The monitored parts can be said patient's limbs (including upper limbs and lower limbs) and/or plantar where the motion signals can be reliably acquired. Multiple parts of the said patient can install measurement modules to acquire the motion signals relevant to the parts. The monitoring patient can be the family member of the patient, the patient's physician or caregiver, or the patient himself. The monitoring patient can carry the said handhold monitoring unit 200 to obtain the information of the motion pattern in real-time from the patient who is been monitored. The said motion patterns include motion type and motion status.

The motion pattern monitoring unit described in the embodiment allows the measurement module to be placed in the part of the patient body where the motion signals can be readily acquired with high signal-to-noise ratio or high fidelity. Based on thus obtained motion signals the peaks of the signals can be detected, and further the peak amplitudes and peak intervals can be derived such that the motion patterns specific to the PD patient can be accurately identified. Eventually the optimized performance in motion patterns monitoring in PD patient can be achieved.

In one embodiment, the said monitoring unit uses the said peak amplitudes and peak interval to identify the said tremor status and/or walking status in the patient.

The said monitoring unit uses the said peak amplitudes and peak intervals to identify the patient's tremor and/or walking status, and save this information together with the time stamps of their occurrences in memory for later analysis or other application.

Tremor and walking have high frequency of occurrence in PD patients, wherein tremor is the most obvious symptom in PD patients; wherein the tremors of PD patient can be resting tremor in which the shaking in the four limbs is visible in resting condition while disappear during sleep or intentionally moving the four limbs, like walking. Tremor has more impact to the distal ends of the four limbs than the proximal ends. In the early phase of the disease, the shaking symptom can only be seen on one hand or one foot. Over time the symptom spreads to the two hands and two feet. The shaking frequency of tremor is within the range of 4 Hz to 6 Hz. The identification of the above-described tremor and walking is of significance in the monitoring of the motion pattern in PD patient.

The embodiment analyzes the motion signals to obtain the signal characteristics such as the peak amplitudes and peak intervals, average and standard deviation of peak amplitude, average peak interval (thus average signal frequency) and standard deviation of the peak intervals, and maximum peak amplitude, in a preset time window. These characteristics of the motion signals are then used in identification of the motion pattern.

As one embodiment, the said monitoring unit acquires the number of tremor and number of steps of walking in a preset time window, and determines the motion pattern of the patient in the preset time window, based on these parameters.

The above-mentioned preset time window can be configured based on the requirement of monitoring, for instance the time window size can be set as 5 seconds. In this case the starting time of the time window is 5 seconds prior to current time.

As one of the embodiments, the said monitoring unit claims detection of the motion pattern in the said preset time window, as: 1) walking and tremor status in the said time window in the said patient, if the said monitoring unit finds that the number of steps in the said time window is greater than or equal to the preset first step threshold and the number of tremors in the said time window is greater than or equal to the preset first tremor threshold; 2) walking status only in the said time window in the said patient, if the said monitoring unit finds that the number of steps in the said time window is greater than or equal to the preset first step threshold and the number of tremors in the said time window is less than the preset first tremor threshold; 3) tremor status only in the said time window in the said patient, if the said monitoring unit finds that the number of steps in the said time window is less than the preset first step threshold and the number of tremors in the said time window is greater than or equal to the preset first tremor threshold; 4) slowness of movement status in the said time window in the said patient, if the said monitoring unit finds that the number of steps in the said time window is greater than the preset second step threshold and less than the preset first step threshold, and the number of tremors in the said time window is greater than the preset second tremor threshold and less than the preset third tremor threshold; and 5) resting status in the said time window in the said patient, if the said monitoring unit finds that the number of steps in the said time window is less than or equal to the preset second step threshold and the number of tremors in the said time window is less than or equal to the preset second tremor threshold; wherein the said first step threshold, the said second step threshold, the said first tremor threshold, the said second tremor threshold and the said third tremor threshold can all be configured based on the size of the time window.

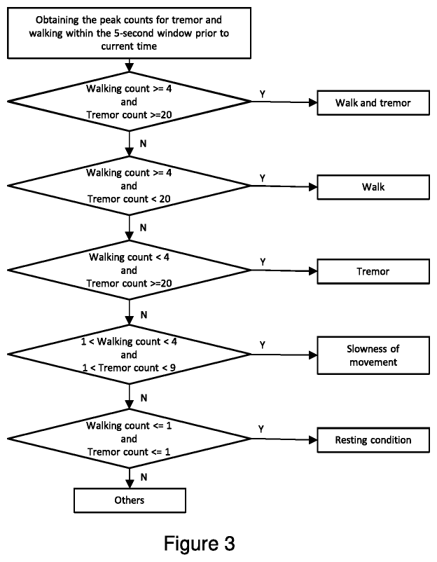

As one of the embodiments, if the size of the time window is set as 5 seconds, then the above first step threshold can be set as 4, the second step threshold set as 1, the first tremor threshold set as 20, the second tremor threshold set as 1, and the third tremor threshold set as 9, then the decision logic to detect the motion patterns can be shown in FIG. 3, including the following processes:

As one of the embodiments, if the preset time window is 5 seconds, then the said first step threshold can be set as 4, the second step threshold as 1, the first tremor threshold as 20, the second tremor threshold as 1 and the third tremor threshold as 9. In this case, the decision logic of motion pattern detection is illustrated in FIG. 3, which includes:

If, the number of steps [symbol] 4, and, the number of tremors<20, the motion pattern is walking only;

If, the number of steps<4, and, the number of tremors [symbol] 20, the motion pattern is tremor only;

If, the number of steps [symbol] 4, and, the number of tremors [symbol] 20, the motion pattern is walking and tremor;

If, 1<the number of steps<4, and, 1<the number of tremors<9, the motion pattern is slowness of movement;

If, the number of steps [symbol] 1, and, the number of tremors [symbol] 1, the motion pattern is resting condition.

It must emphasize that, firstly, although there is the word “tremor” in the term “the number of tremors”, that does not necessarily mean there is tremor. Instead, it actually means the number of peaks that falling in the specific bandwidth; secondly, over time with the increase of data acquired and accumulation of experience, in the actually implementation of the algorithm in product, the above-mentioned thresholds may be tuned and the decision logic be slightly adjusted in order to optimize the performance of the motion pattern identification.

In one of the embodiments, the said measurement modules include the first measurement module;

wherein the said first measurement module acquires the acceleration signals from the wrist of the patient and transmits them to the said monitoring unit.

In the current embodiment, the said motion signals are the acceleration signals acquired from the wrist of the patient. When the patient is in walking or tremor status, the characteristic motion at the wrist is relatively distinct. Therefore, placing the said first measurement module at the wrist of the patient makes the motion patterns expressed in the acceleration signals easier to identify.

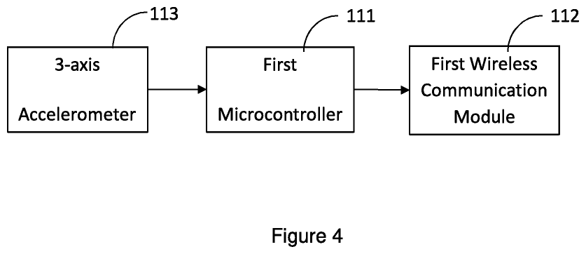

As one of the embodiments, as FIG. 4 shows, the said first measurement module consists of the first microcontroller 111, the first wireless communication module 112 and the accelerometer 113.

The said accelerometer 113 is connected to the first wireless communication module 112 through the said first microcontroller 111.

The said accelerometer 113 acquires the acceleration signals on the wrist of the patient and transmits the signals to the said microcontroller 111;

The said first microcontroller 111 further transmits the acceleration signals to the said monitor unit.

The said first wireless communication module 112 may contain communication module, such as Bluetooth communication module, to implement the wireless communication functionality. The said accelerometer 113 may consist of multiple sensors, each of the sensors is connected to the said first microcontroller 111, to which the acquired acceleration signals are transmitted. In other words, accelerometers can be placed on both wrists of the patient such that the integrity of the motion patterns can be better preserved in the motion signals.

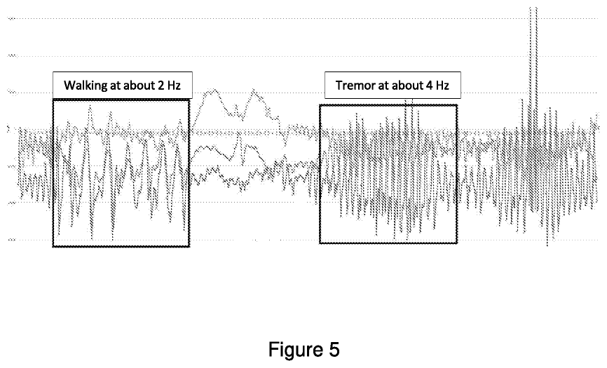

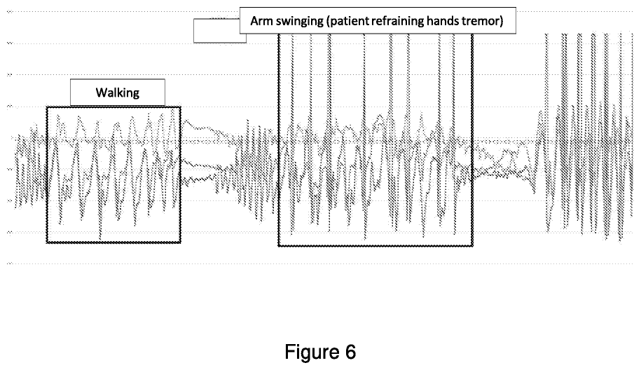

As one of the embodiments, the said accelerometer can be 3-D accelerometer, the advantage of which is the small size, lower power consumption and lower cost. The 3-D accelerometer mounted in a wristwatch-like device can be used to record the motion signals generated by the limb movement and/or torso movement in the PD patient. The acceleration signals acquired by the 3-D accelerometer can be decomposed into DC component and AC component, in which the DC component corresponds to non-rhythmic movement of the torso, while the AC component corresponds to the rhythmic movement generated by both torso and upper limbs, such as tremor in PD patient. PD patient can wear the wristwatch-like device installed with 3-D accelerometer on either left wrist, or right wrist, or both wrists. The exemplary acceleration signals recorded by such a wrist worn device is shown in FIG. 5, where walking is presented by low frequency signals, while tremor is presented by high frequency signals. The motion pattern related information is not only embedded in frequency of the acceleration signals, but also in amplitude. FIG. 6 presents the typical acceleration signals when the PD patient naturally swing their arms during walking and deliberately swing their aim in order to suppress the tremor.

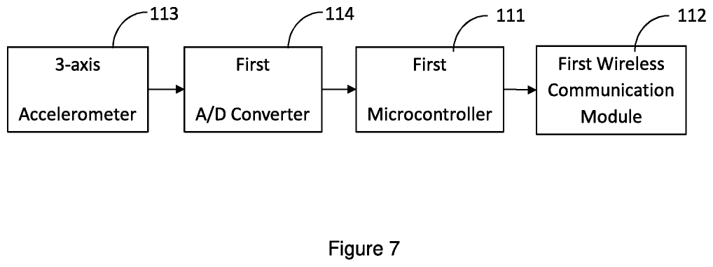

As one of the embodiments, as shown in FIG. 7, the said first measurement module also comprises the first analog-to-digital converter (i. e. ADC) 114; wherein the said first ADC 114 connects the accelerometer 113 to the said first microcontroller 111.

In this embodiment, the acceleration signals acquired by the said first accelerometer 113 are transmitted to the said first ADC 114 and are converted to the digital signals for later transmission. For digital signal communication are usually more reliable in dealing with electromagnetic interference.

In one of the embodiments, the said measurement module may have a second measurement module;

wherein the said second measurement module is placed under the plantar of the patient;

wherein the said second measurement module is also wireless connected to the said monitoring unit;

wherein the said second measurement module acquires the plantar pressure signals from the plantar of the patient and transmits the plantar pressure signals to the said monitor unit.

In the embodiment, the said motion signals are the plantar pressure signals acquired from the plantar of the patient. When the use walks or tremor occurs, distinct motions can be seen in the foot of the PD patient. Therefore, the plantar pressure signals acquired from the said second measurement module placed under the plantar of the patient can effectively illustrates the motion characteristics of the patient.

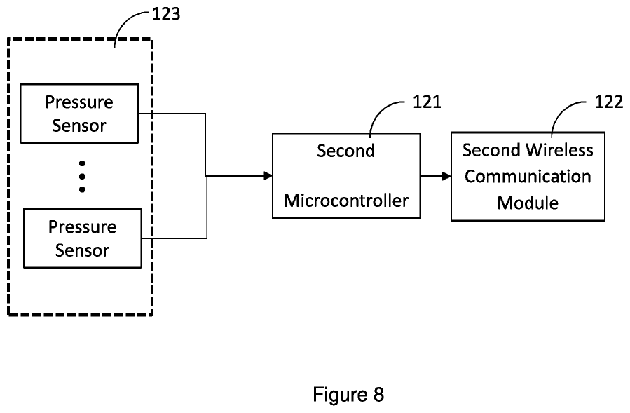

As one of the embodiments, as shown in FIG. 8, the said second measurement module includes a second microcontroller 121, a second wireless communication unit 122 and multiple pressure sensors 123.

wherein each of the pressure sensors 123 is connected to the said second wireless communication module 122 via the said second microcontroller 121;

wherein the said pressure sensors 123 acquire the plantar pressure signals when the patient moves, and the plantar pressure signals are transmitted to the said second microcontroller 121;

wherein the said second microcontroller 121 transmits the plantar pressure signals through the said second wireless communication module to the said monitoring unit.

The above-mentioned second wireless communication module 122 may be implemented with Bluetooth communication module. Also, the above-mentioned pressure sensors can be placed under both plantares of the patient such that the integrity of the motion patterns can be better preserved in the pressure signals.

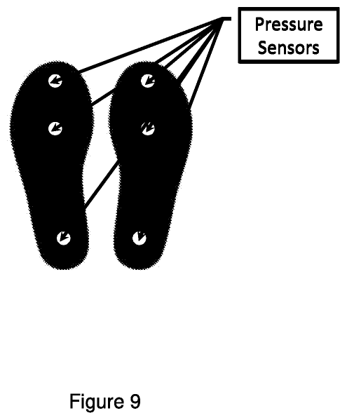

As one of the embodiments, the above-mentioned pressure sensors can consist of 6 sensors, 3 sensors placed in each of the plantar. The schematic diagram of the configuration of the plantar pressure sensor can be found in FIG. 9, where one of the pressure sensors is placed under heel or posterior support area, a second under foot bow or lateral support area, and a third under the anterior support area. This configuration on one hand covers the three most important areas of a foot, on the other hand simplifies the number of the sensors used in the data acquisition in the said second measurement module. Upon the acquisition of the plantar pressure signals from the plantar pressure sensors, motion patterns of the patient can be identified based on the analysis of the plantar pressure signals. For instance, when the patient has abnormal gait, the force applied to the heel appears to be bigger, and it takes a longer time (e.g. 17 seconds) to decelerate in order to sit down from standing. Therefore, the application of the plantar pressure sensors in the monitoring of motion patterns of the PD patient can reveal not only the gait and posture, but also various types of physical activity.

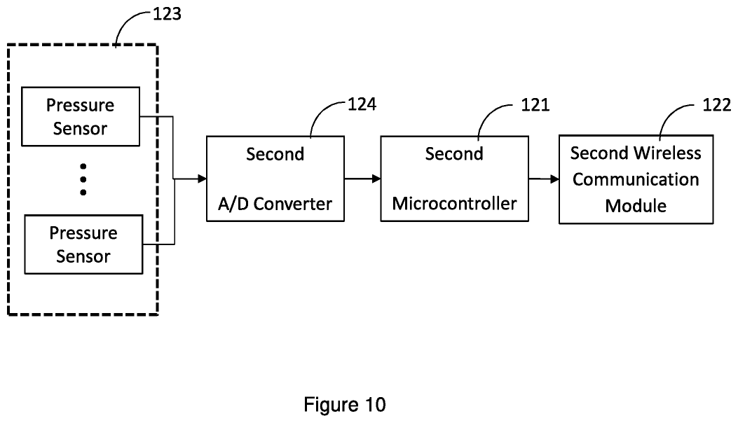

As one of the embodiments, as shown in FIG. 10, the said second measurement module also consists of a second ADC 124; wherein the inputs of the said second ADC 124 are connected to the pressure sensors; wherein the outputs of the said second ADC are connected to the said second microcontroller 121.

In this embodiment, the pressure signals acquired by the said pressure sensors 123 are transmitted to the said ADC 1244 and are converted to the digital signals for later transmission. For digital signal communication are usually more reliable in dealing with electromagnetic interference.

In one of the embodiments, the above-mentioned patient is a PD patient, the above-mentioned measurement module may include a first measurement module and a second measurement module; for instance, based on the situation of the PD patient the first measurement module can be selected as a module placed on wrist to acquire acceleration signals of the wrist, and/or the second measurement module be selected as a module placed under the plantar of the PD patient to acquire the patient's plantar pressure signals. For example, if the PD patient has only tremor in upper limbs, then only one first measurement module is used; if the PD patient has only tremor in lower limbs, then only the second measurement module is used; otherwise both first measurement module and second measurement module are used.

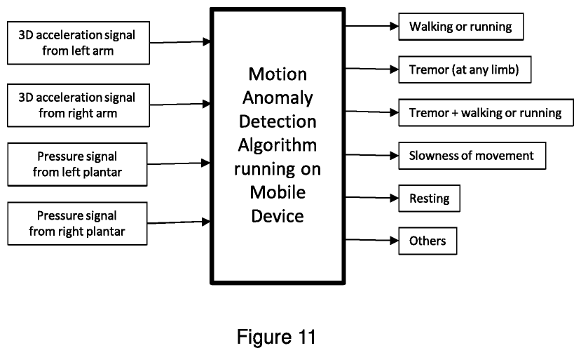

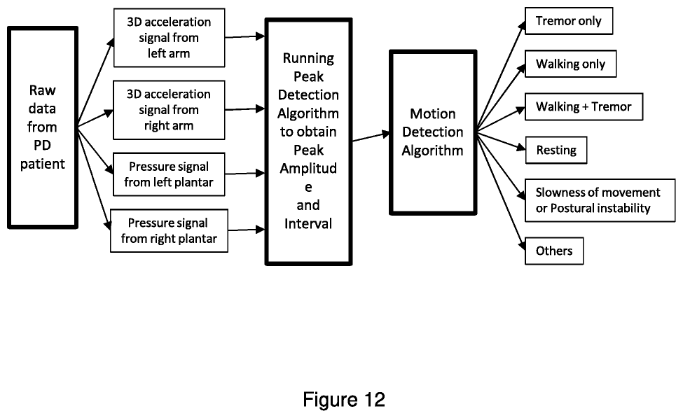

As one of the embodiments, the above-mentioned monitoring unit is a mobile device; wherein the said first measurement module consists of multiple 3-D accelerometers; wherein said 3-D accelerometers are placed on both wrists of the PD patient in order to acquire the acceleration signals on both left arm and right arm; wherein the pressure sensors of the said second measurement modules are placed under both plantares in order to acquire the pressure signals from left foot and right foot; wherein the mobile device, as shown in FIG. 11, receives the data transmitted from the measurement modules and analyzes the signals to identify the motion patterns of the PD patient, such as limb tremor, walking or running, limb tremor plus walking or running, slowness of movement and resting condition, etc. The motion pattern information can be displayed on the mobile device for the PD patient and the physician to check the patient's motion status, and accordingly to adjust the behavior of the PD patient and/or treatment. As a result, the PD patient can be effectively managed. The monitoring process of the motion pattern of the PD patient is shown in FIG. 12; wherein the 3-D acceleration signals from left arm, 3-D acceleration signals from right arm, plantar pressure signals from left plantar and plantar pressure signals from right plantar are all acquired and analyzed in order to achieve full scope monitoring of the patient's motion status.

The technical features of the above-described embodiments can be combined in various ways. For the purpose of concise description, not all the possible combination of the technical features of the embodiments are covered. However, if there is no contradiction between the combinations of the technical features, all should be considered be within the range of the disclosure.

The above-described embodiments, of which the descriptions are very concrete and detailed, only cover a few implementations of the invention. This cannot be considered as the limitation to the range of the invention. It must point out that, for one skilled with the art, many modifications and improvements, which are protected by the invention, can be made without departing from the conception of the invention. Therefore, the scope of protection of the present invention shall be subject to the claims.

1. An apparatus for detection of motion pattern, comprising:
measurement modules and a monitoring unit;
The said measurement modules are placed to the parts of the patient body that are to be measured; and the said monitoring unit is a handhold unit carried by the patient; and when the said measurement modules acquire the motion signals from the said patient, the said motion signals are transmitted to the said monitoring unit;
The measurement modules measure the said patient's motion signals and sends the said motions signals to the said monitoring unit;
The said monitoring unit identifies each of the peaks and find the peak amplitudes in the said motion signals, calculates the intervals between each of two consecutive peaks, and monitors the motion pattern of the said patient based on the said peak amplitudes and the said peak intervals.

2. Based on claim 1 of the apparatus for detection of motion pattern, the said monitoring unit identifies the tremor status and/or walking status in the said patient based on the said peak amplitudes and the said peak intervals.

3. Based on claim 2 of the apparatus for detection of motion pattern, the said monitoring unit obtains the number of peaks of tremor signal component (hereafter called number of tremors) and the number of peaks of walking signal component (hereafter called number of steps) that the said patient completes in a preset time window, and uses these two numbers to monitor the motion status of the said patient in the said preset time window.

4. Based on claim 3 of the apparatus for detection of motion pattern, the said monitoring unit claims detection of: 1) walking and tremor status in the said time window in the said patient, if the said monitoring unit finds that the number of steps in the said time window is greater than or equal to the preset first step threshold and the number of tremors in the said time window is greater than or equal to the preset first tremor threshold; 2) walking status only in the said time window in the said patient, if the said monitoring unit finds that the number of steps in the said time window is greater than or equal to the preset first step threshold and the number of tremors in the said time window is less than the preset first tremor threshold; 3) tremor status only in the said time window in the said patient, if the said monitoring unit finds that the number of steps in the said time window is less than the preset first step threshold and the number of tremors in the said time window is greater than or equal to the preset first tremor threshold; 4) slowness of movement status in the said time window in the said patient, if the said monitoring unit finds that the number of steps in the said time window is greater than the preset second step threshold and less than the preset first step threshold, and the number of tremors in the said time window is greater than the preset second tremor threshold and less than the preset third tremor threshold; and 5) resting status in the said time window in the said patient, if the said monitoring unit finds that the number of steps in the said time window is less than or equal to the preset second step threshold and the number of tremors in the said time window is less than or equal to the preset second tremor threshold.

5. Based on claim 1 of the apparatus for detection of motion pattern, the measurement modules comprise a first measurement module; the said first measurement module is placed on the wrist of the said patient; the said first measurement module is connected to the monitoring unit wirelessly; the said first measurement module acquires the 3-D acceleration signals at the wrist of the patient and transmits the 3-D acceleration signals to the said monitoring unit.

6. Based on claim 5 of the apparatus for detection of motion pattern, the said first measurement module comprises a first microcontroller, a first wireless communication module and a 3-D accelerometer sensor; the said 3-D accelerometer sensor is connected to the said first wireless communication module through the said first microcontroller; the acceleration signals at the said wrist of the said patient are acquired by the said 3-D accelerometer sensor and are transmitted to the said first microcontroller; the said first microcontroller transmits the 3-D acceleration signals to the said monitoring unit through the said first wireless communication module.

7. Based on claim 6 of the apparatus for detection of motion pattern, the said first measurement module also comprises a first Analog-to-Digital converter (hereafter called ADC); the ADC connects the said 3-D accelerometer sensor to the said first microcontroller.

8. Based on claim 1 of the apparatus for detection of motion pattern, the said measurement modules comprises a second measurement module; the said second measurement module is placed under the plantar of the said patient; the said second measurement module is connected to the said monitoring unit through wireless communication methods; the plantar pressure signals acquired by the said second measurement module are wirelessly transmitted to the said monitoring unit.

9. Based on claim 8 of the apparatus for detection of motion pattern, the said second measurement module comprises a second microcontroller, a second wireless communication module and multiple pressure sensors; each of the plantar pressure sensors are connected to the said wireless communication module through the said second wireless microcontroller; the said plantar pressure signals acquired by the said plantar pressure sensors are transmitted to the said monitoring unit through the said second microcontroller.

10. Based on claim 9 of the apparatus for detection of motion pattern, the said second measurement module comprises a second ADC; the inputs of the said second ADC are connected to each of the said plantar pressure sensors; the output of the said second ADC are connected to the said second microcontroller.
